(19) United States
(12) Patent Application Publication
(10) Pub. No.: US 2018/0017485 A1
(43) Pub. Date: Jan. 18, 2018

(54) SENSOR

(71) Applicant: Panasonic Intellectual Property Management Co., Ltd., Osaka (JP)

(72) Inventors: KOJI SAKAI, Hyogo (JP); SHINICHI KISHIMOTO, Osaka (JP); DAISUKE WAKABAYASHI, Fukui (JP)

(21) Appl. No.: 15/542,411

(22) PCT Filed: Jan. 20, 2016
(86) PCT No.: PCT/JP2016/000263

§ 371 Date: Jul. 7, 2017

(30) Foreign Application Priority Data
Jan. 29, 2015 (JP) 2015-014880
Jan. 29, 2015 (JP) 2015-014881
Jan. 29, 2015 (JP) 2015-014882

(51) Int. Cl. G01N 21/3577; G01N 21/01; G01N 21/27; G01N 21/31; G01N 33/28; G01N 21/59

(52) U.S. Cl. CPC G01N 21/3577; G01N 21/31; G01N 33/2835; G01N 21/01; G01N 21/27; G01N 2021/5961

(57) ABSTRACT

A purpose of the present invention is to provide a sensor with high sensitivity or high target substance selectivity.

A sensor that includes a structure having an internal space into which a detection target is capable of flowing, a light-emitting element, and a photo-receptor element is provided. The sensor is disposed so that light emitted from the light-emitting element passes through the internal space to reach the photo-receptor element. A wavelength of the light emitted from the light-emitting element falls within a range from 2.5 μm to 15 μm inclusive. A length of the internal space in a direction perpendicular to an extension direction of the structure should preferably be less than or equal to 1000 μm.

### TECHNICAL FIELD

The present invention relates to a sensor for use in, for example, a fluid component detection device for detecting density of a fluid component by using a property of absorbing light such as infrared light.

### BACKGROUND ART

Conventional fluid component detection devices include, for example, devices disclosed in PTLs 1 to 6. In techniques common to PTLs 1 to 6, a vessel for storing a fluid containing a detection target is disposed between a light-emitting portion and a photo-receptor portion. For light such as infrared light irradiated from the light-emitting portion, in accordance with an amount of light such as infrared light that is not absorbed by a fluid component that is a detection target but that is received by the photo-receptor portion, density of the fluid component in a vessel can be detected.

### CITATION LIST

### Patent Literature

PTL 1: Unexamined Japanese Patent Publication No. 2010-145252

PTL 2: Unexamined Japanese Patent Publication No. 2010-145107

PTL 3: Unexamined Japanese Patent Publication No. 2009-36746

PTL 4: Japanese Translation of PCT International Application Publication No. 2007-528982

PTL 5: Unexamined Japanese Patent Publication No. 1997-229847

PTL 6: Unexamined Japanese Patent Publication No. 1995-294428

### SUMMARY OF THE INVENTION

However, with sensors according to PTLs 1 to 6, a sensor with high sensitivity or high target substance selectivity would not likely be fully provided.

In view of the above problem in the conventional art, an object of the present invention is to provide a sensor with high sensitivity or high target substance selectivity.

A sensor according to the present invention includes a structure having an internal space into which a detection target is capable of flowing, a light-emitting element, and a photo-receptor element. The sensor is disposed so that light emitted from the light-emitting element passes through the internal space to reach the photo-receptor element, and a wavelength of the light emitted from light-emitting element falls within a range from 2.5 μm to 15 μm inclusive. Light having the above described wavelength can easily be absorbed by the detection target, and the light makes the detection target show a steep spectral characteristic. Therefore, the sensor with high sensitivity and high selectivity can be provided.

In addition, a sensor of the present invention includes a structure having an internal space into which a detection target is capable of flowing, a light-emitting element, and a photo-receptor element. The sensor is disposed so that light emitted from the light-emitting element passes through the internal space to reach the photo-receptor element, and the structure is made up of a semiconductor substrate. Since the structure is made up of a semiconductor substrate, the sensor can easily be small-sized, and thus the sensor with high sensitivity and high selectivity can be provided.

In addition, a sensor of the present invention includes a structure having an internal space into which a detection target is capable of flowing, a light-emitting element, and a photo-receptor element. The sensor is disposed so that light emitted from the light-emitting element passes through the internal space to reach the photo-receptor element, and the structure has a function capable of heating the detection target flowed into the internal space. Since the structure has the function capable of heating a detection target, even if the sensor is small-sized and accordingly an internal space is narrowed, a detection target flowed into the internal space can be heated, and thus a convective flow is generated. This enables the detection target to easily enter and exit into and from the internal space. As a result, the sensor with high sensitivity and high target substance selectivity can be provided.

According to the present invention, a sensor with high sensitivity or high target substance selectivity can be provided.

### BRIEF DESCRIPTION OF DRAWINGS

FIG. 1 is a schematic perspective view of a sensor according to an exemplary embodiment.

FIG. 2 is a top view of the sensor according to the exemplary embodiment when disposed in a pipe.

FIG. 3 is a schematic plan view illustrating an optical path and a disposition relationship of a structure, a light-emitting element, and a photo-receptor element configuring the sensor according to the exemplary embodiment.

FIG. 4 is a schematic cross-sectional view illustrating the optical path and the disposition relationship of the structure, the light-emitting element, and the photo-receptor element configuring the sensor according to the exemplary embodiment.

FIG. 5 is a schematic plan view illustrating an optical path and a disposition relationship of a structure, a light-emitting element, and a photo-receptor element configuring a sensor according to a first modification to the exemplary embodiment.

FIG. 6 is a schematic cross-sectional view of the structure configuring the sensor according to the exemplary embodiment.

FIG. 7 is a schematic cross-sectional view of a structure configuring a sensor according to a second modification to the exemplary embodiment.

FIG. 8A is a top view illustrating an example of the light-emitting element configuring the sensor according to the exemplary embodiment.

FIG. 8B is a cross-sectional view illustrating the example of the light-emitting element configuring the sensor according to the exemplary embodiment.

FIG. 9A is a top view illustrating an example of the photo-receptor element configuring the sensor according to the exemplary embodiment.

FIG. 9B is a cross-sectional view illustrating the example of the photo-receptor element configuring the sensor according to the exemplary embodiment.

FIG. 10 is a schematic cross-sectional view of a structure configuring a sensor according to a third modification to the exemplary embodiment.

FIG. 11 is a schematic cross-sectional view of a structure configuring a sensor according to a fourth modification to the exemplary embodiment.

FIG. 12 is a schematic plan view illustrating an optical path and a disposition relationship of a structure, a light-emitting element, and a photo-receptor element configuring a sensor according to a fifth modification to the exemplary embodiment.

FIG. 13 is a schematic plan view illustrating an optical path and a disposition relationship of a structure, a light-emitting element, and a photo-receptor element configuring a sensor according to a sixth modification to the exemplary embodiment.

FIG. 14 is a schematic plan view illustrating an optical path and a disposition relationship of a structure, a light-emitting element, and a photo-receptor element configuring a sensor according to a seventh modification to the exemplary embodiment.

### DESCRIPTION OF EMBODIMENT

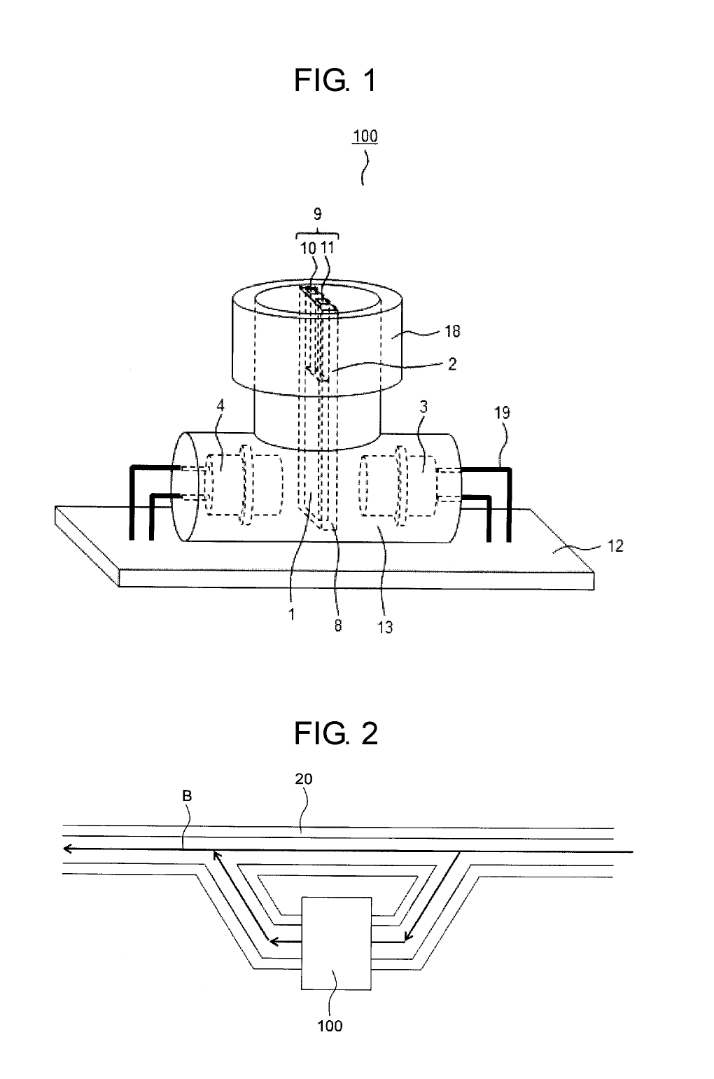

A sensor according to an exemplary embodiment of the present invention will now be described herein with reference to FIGS. 1 to 14. In FIGS. 1 to 14, an identical component is applied with an identical numeral or symbol, and its description might be sometimes appropriately omitted. In addition, FIGS. 1 to 14 illustrate examples of advantageous exemplary embodiment and modifications, and the present invention is not limited to respective forms. In addition, features illustrated in the drawings can be combined as long as a contradiction will not arise.

### Exemplary Embodiment

A sensor according to an exemplary embodiment will now be described herein with reference to FIG. 1.

As illustrated in FIG. 1, sensor 100 includes structure 2 having internal space 1 into which a detection target is capable of flowing, light-emitting element 3, and photo-receptor element 4, where sensor 100 is disposed so that light emitted from light-emitting element 3 passes through the internal space to reach photo-receptor element 4, and a wavelength of the light emitted from light-emitting element 3 falls within a range from 2.5 μm to 15 μm inclusive.

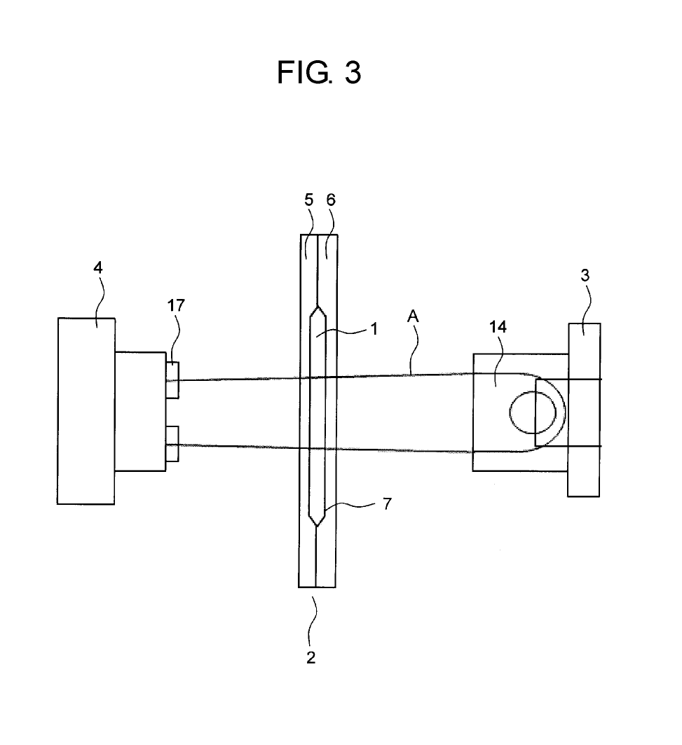

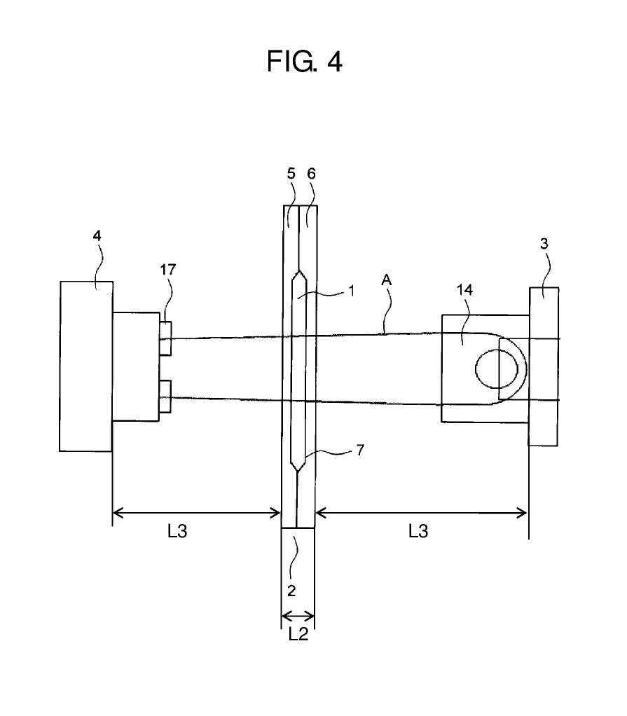

According to this configuration, as illustrated in FIGS. 3, 4, light A emitted from light-emitting element 3 penetrates through structure 2, and enters into internal space 1. Light A penetrates through internal space 1 and structure 2 to reach photo-receptor element 4. At this time, a fluid containing a detection target present in internal space 1 absorbs the light, an amount of light received by photo-receptor element 4 reduces, and, in accordance with the amount of light received, a signal processing circuit portion signal-processes an output signal from photo-receptor element 4. Therefore, density of the detection target contained in the fluid in internal space 1 can be detected. Since light-emitting element 3 and photo-receptor element 4 lie outside structure 2, the fluid filled in internal space 1 can be prevented from directly coming into contact with light-emitting element 3 and photo-receptor element 4. Therefore, light-emitting element 3 and photo-receptor element 4 can be prevented from being contaminated with particles in the fluid. In addition, a thickness of structure 2 can be reduced, and thus a whole sensor can be small-sized. In addition, since a wavelength of the light emitted from light-emitting element 3 falls within a range from 2.5 μm to 15 μm inclusive, the light can be absorbed by the detection target more easily than a case when light having a wavelength below 2.5 μm is used, and thus the detection target will show a steep spectral characteristic. Therefore, the sensor with high sensitivity and high target substance selectivity can be provided.

As illustrated in FIGS. 1, 2, sensor 100 is coupled to pipe 20 via pipe bonding portion 18. As illustrated in FIGS. 1, 2, fluid B passing through pipe 20 enters from inflow port 10 of sensor 100, passes through internal space 1, and exits from outflow port 11 to return to pipe 20. As a fluid passing through pipe 20, a fuel for a vehicle may be used. A fuel consists of hydrocarbon system components, ethanol, water, and other materials, and the hydrocarbon system components include an aroma system, an olefin system, a paraffin system, and other similar systems. With sensor 100, density of these fuel components can be detected, and, for example, in an internal combustion engine, fuel efficiency can be improved, and exhaust emissions can be reduced.

In addition, as illustrated in FIG. 1, sensor 100 further includes printed substrate 12, where light-emitting element 3, photo-receptor element 4, and structure 2 are sealed by sealing body 13, and sealing body 13 is mounted on printed substrate 12. Since a pipe to be disposed with sensor 100 might be disposed near an engine, in such a case, the pipe is required to be robust so as to withstand vibrations of the engine. Sealing body 13 may be made of a resin. In addition, light-emitting element 3, photo-receptor element 4, and structure 2 may be fitted each other to configure sealing body 13.

As illustrated in FIGS. 3, 4, structure 2 includes first substrate 5 and second substrate 6, where first substrate 5 and second substrate 6 are bonded each other at respective peripheral portions of internal space 1. A method for forming internal space 1 will now briefly be described herein. First, first substrate 5 and second substrate 6 are prepared. Next, both of first substrate 5 and second substrate 6 are immersed in an etching solution to form groove 7 through etching. Next, first substrate 5 and second substrate 6 are bonded at the respective peripheral portions so that a first groove formed on first substrate 5 and a second groove formed on second substrate 6 face each other. With the above described manufacturing method, internal space 1 is formed. However, a groove should not always be formed on both of first substrate 5 and second substrate 6. For example, internal space 1 may be made up of only the first groove disposed in first substrate 5, or may be made up of only the second groove disposed in second substrate 6. In addition, groove 7 may preferably be shaped so as to be narrowed toward an advancement direction of etching. However, the present invention is not limited to this shape. First substrate 5 should preferably be made up of a semiconductor substrate that allows infrared light to penetrates through, such as silicon and germanium, and second substrate 6 should preferably be made up of a semiconductor substrate that allows infrared light to penetrate through, such as silicon and germanium. As described above, first substrate 5 and second substrate 6 should preferably be made up of an identical material. However, the present invention is not limited to this configuration. For example, first substrate 5 may be made up of glass, and second substrate 6 may be made up of a semiconductor substrate such as a silicon substrate. In addition, as other examples of first substrate 5 and second substrate 6, a material having an optically transparent feature with respect to light having a wavelength in a range from 2.5 μm to 15 μm inclusive may be used. For example, sapphire, zinc selenide, a resin material, or another material may be used. These materials can appropriately be selected by taking into account a manufacturing process, a cost, and other factors. First substrate 5 and second substrate 6 may preferably be directly bonded each other without a bonding material being interposed at the respective peripheral portions of internal space 1. However, the present invention is not limited to this bonding method. Examples of directly bonding methods include, for example, low temperature direct bonding such as surface activated bonding. Such a method can reduce an internal stress along with bonding of first substrate 5 and second substrate 6. However, first substrate 5 and second substrate 6 may be bonded each other with a bonding material being interposed. In this case, as a bonding material, a resin material, a solder material, or an alloy of gold and tin may be used.

As illustrated in FIGS. 3, 4, when viewed in a direction perpendicular to an extension direction of structure 2, internal space 1, light-emitting element 3, and photo-receptor element 4 should preferably be disposed so as to overlap each other. However, the present invention is not limited to this disposition. For example, as long as a direction of light from light-emitting element 3 toward internal space 1 is inclined with respect to a bottom face of structure 2, internal space 1 and light-emitting element 3 can be disposed so as not to overlap each other when viewed from the direction perpendicular to the extension direction of structure 2. Similarly, as long as a direction of light from internal space 1 toward photo-receptor element 4 is inclined with respect to the bottom face of structure 2, internal space 1 and photo-receptor element 4 can be disposed so as not to overlap each other when viewed from the direction perpendicular to the extension direction of structure 2.

A thickness of structure 2 (a length in the direction perpendicular to the extension direction of the structure) should preferably fall within a range from 450 μm to 1350 μm inclusive. However, the present invention is not limited to this thickness. In addition, first substrate 5 should preferably have a thickness within a range from 350 μm to 800 μm inclusive. However, the present invention is not limited to this thickness. In addition, second substrate 6 should preferably have a thickness within a range from 100 μm to 550 μm inclusive. However, the present invention is not limited to this thickness. In addition, for thicknesses of first substrate 5 and second substrate 6, in light of a smaller sensor and in light of securing an enough internal space, either of the substrates, which is formed with a thicker groove, should preferably be thicker than another of the substrates. In addition, a thickness of internal space 1 should preferably be less than or equal to 1000 μm. Further, the thickness should preferably fall within a range from 250 μm to 500 μm inclusive. However, the present invention is not limited to the values. In addition, a thickness from a top face of internal space 1 to a top face of structure 2 should preferably fall within a range from 100 μm to 300 μm inclusive. However, the present invention is not limited to the values.

A length of internal space 1 in the direction perpendicular to the extension direction of structure 2 should preferably be less than or equal to 1000 μm. Alternatively, a length of structure 2 in a direction toward which light penetrates through should preferably be less than or equal to 1000 μm. Since a wavelength of light emitted from light-emitting element 3 falls within a range from 2.5 μm to 15 μm inclusive, light reaching photo-receptor element 4 would easily be absorbed by a detection target, and thus would easily be attenuated. Therefore, in order to prevent an amount of light passing through from decreasing to a detection limit or below, an optical path should preferably be shortened. As described above, in internal space 1, an optical path length of light should preferably be less than or equal to 1000 μm.

As illustrated in FIGS. 3, 4, thickness L2 of structure 2 should preferably be smaller than distance L3 between structure 2 and light-emitting element 3 or photo-receptor element 4. However, the present invention is not limited to this distance. In addition, as illustrated in FIGS. 3, 4, a length of structure 2 in a direction in parallel to a straight direction of light should preferably be shorter than a length of light-emitting element 3 in a straight direction of light. A balance between small sizing and an optical characteristic should be taken into account.

In addition, as illustrated in FIGS. 3, 4, the extension direction of internal space 1 is in parallel to the extension direction of structure 2. By increasing a distribution ratio of an internal space in a structure, an unnecessary region in the structure can be reduced, and thus a whole sensor can be small-sized.

In addition, as illustrated in FIGS. 3, 4, in order to allow light emitted from light-emitting element 3 to pass through structure 2 to reach photo-receptor element 4, structure 2 lies between light-emitting element 3 and photo-receptor element 4.

In addition, as illustrated in FIG. 4, structure 2 includes first end 8 in the extension direction of internal space 1, and second end 9 on a side of structure 2 opposite from first end 8, where first end 8 is closed, and second end 9 is open so that a detection target can enter and exit. Second end 9 includes inflow port 10 and outflow port 11 for a detection target. Inflow port 10 and outflow port 11 can also be shared, but, by separating opening portions, a fluid containing a detection target can easily reach first end 8 of internal space 1. Therefore, even when a distance between first end 8 and second end 9 is longer, a greater effect of bringing a fluid containing a detection target in particular toward first end 8 can be achieved.

In addition, as illustrated in FIG. 4, a distance from light-emitting element 3 to first end 8 of structure 2 is shorter than a distance from light-emitting element 3 to second end 9 of structure 2. The internal space is wider at around an area near second end 9 than at an area near first end 8. Therefore, positioning for disposition in order to allow light emitted from light-emitting element 3 to pass through the internal space can easily be performed.

In addition, as illustrated in FIGS. 3, 4, reflecting mirror 14 capable of concentrating light emitted from light-emitting element 3 is included. In addition, although not illustrated in the drawings, in order to concentrate light emitted from light-emitting element 3, a lens may be provided between structure 2 and light-emitting element 3. By increasing an intensity of light, a sensor with high sensitivity and high target substance selectivity can be provided.

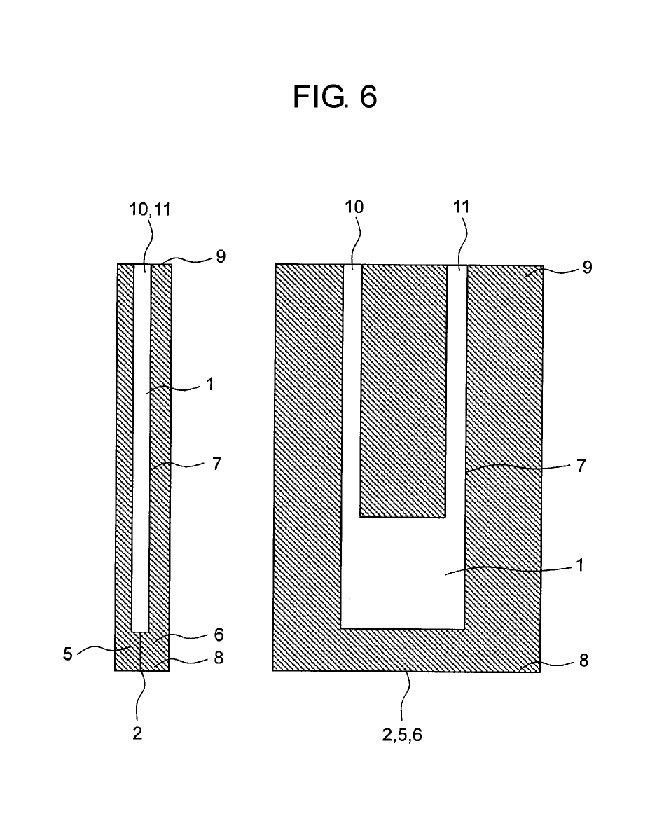

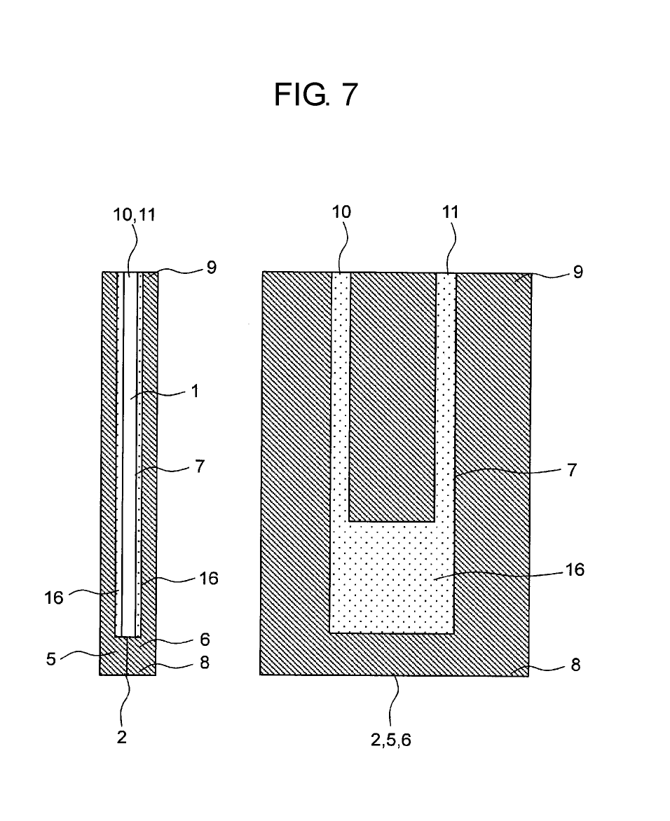

In addition, as illustrated in FIG. 6, internal space 1 is formed by groove 7 of structure 2, and nothing may be formed on groove 7. On the other hand, as illustrated in FIG. 7, internal space 1 is formed by groove 7 of structure 2, and, on a side of groove 7, which lies closer to light-emitting element 3, anti-reflection film 16 may be disposed. Anti-reflection film 16 may further be disposed on another side lying closer to photo-receptor element 4. With anti-reflection film 16, through a surface reflection due to differences in refraction factor among a member configuring structure 2, air, and a fluid in internal space 1, an amount of light to reach photo-receptor element 4 can be prevented from reducing. A left view of FIG. 6 is a cross-sectional front view of structure 2. A right view of FIG. 6 is a cross-sectional side view of structure 2. A left view of FIG. 7 is a cross-sectional front view of structure 2 according to a modification. A right view of FIG. 7 is a cross-sectional side view of structure 2 according to the modification.

In addition, as illustrated in FIGS. 3, 4, two or more optical filters 17 each having a different transmission wavelength are disposed between structure 2 and photo-receptor element 4, and light from light-emitting element 3 penetrates through optical filters 17 to reach photo-receptor element 4. Optical filters 17 may be disposed between structure 2 and light-emitting element 3. Optical filters 17 should respectively preferably be made up of a band pass filter made of a dielectric multi-layer film having a pass band including a wavelength band of light to be absorbed by a detection target.

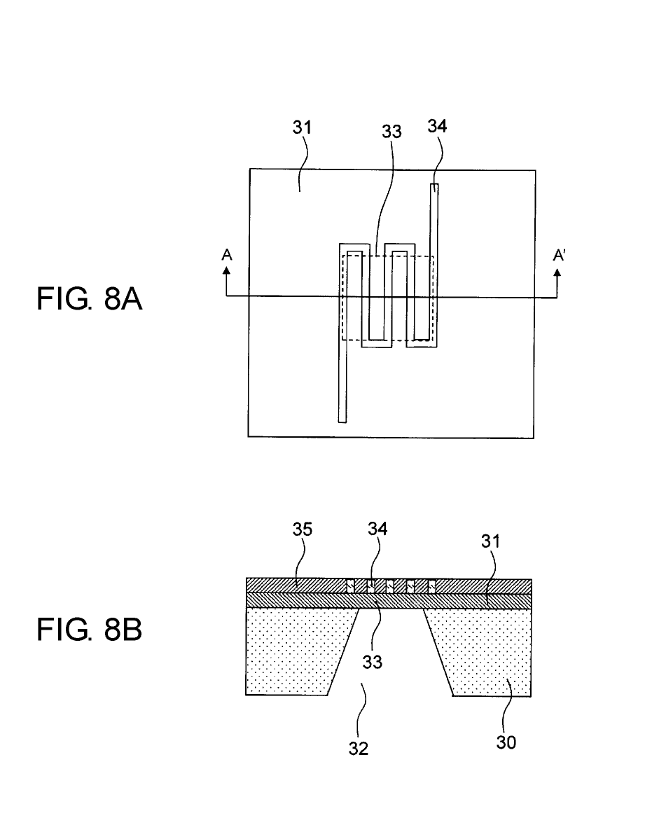

In addition, light-emitting element 3 may be made up of, for example, a light emitting diode, or, as illustrated in FIGS. 8A, 8B, may be made up of a Micro Electro Mechanical Systems (MEMS) chip (a chip formed through a semiconductor micro-machining process) based mainly on a material such as a semiconductor substrate. FIG. 8A is a top view of a light-emitting element made up of an MEMS chip, and FIG. 8B is a cross-sectional view taken along line A-A′ illustrated in FIG. 8A. To produce the light-emitting element made up of the MEMS chip, for example, as illustrated in FIGS. 8A, 8B, recessed portion 32 is provided, by using an etching solution such as Tetramethylammonium hydroxide (TMAH), on a lower face of a structure in which semiconductor substrate 30 such as a silicon substrate and insulating layer 31 such as a silicon oxide film are laminated, and thus diaphragm portion 33 is formed on a top portion of semiconductor substrate 30. Light emission regions 34 made of a metal such as platinum are formed on diaphragm portion 33 via insulating layer 31 such as a silicon oxide film, and further insulating layer 35 is formed.

Light-emitting element 3 may include two or more light sources each having a different wavelength. When light sources each having a narrower wavelength, such as a Light Emitting Diode (LED), are used, the light sources each having a different wavelength are arranged (arrayed) two-dimensionally and horizontally. Irradiation of light having a plurality of types of wavelengths enables detection of a plurality of types of detection targets. In this case, wavelengths of the light emitted variously from a plurality of light-emitting elements are all fall within a range from 2.5 μm to 15 μm inclusive. Therefore, with high sensitivity and high target substance selectivity kept maintained, a plurality of types of detection targets can be detected.

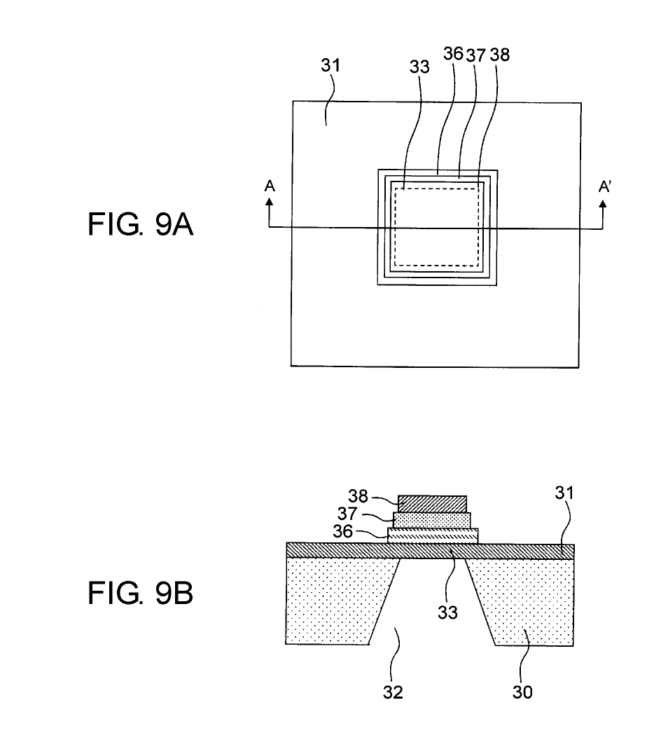

In addition, photo-receptor element 4 may be made up of, for example, a photo diode, or, as illustrated in FIGS. 9A, 9B, may be made up of an MEMS chip such as a pyroelectric element mainly made up of a material such as a semiconductor substrate. FIG. 9A is a top view of a photo-receptor element made up of an MEMS chip, and FIG. 9B is a cross-sectional view taken along line A-A′ in FIG. 9A. To produce the photo-receptor element made up of the MEMS chip, as illustrated in FIGS. 9A, 9B, recessed portion 32 is provided, by using an etching solution such as TMAH, on a lower face of a structure in which semiconductor substrate 30 such as a silicon substrate and insulating layer 31 such as silicon oxide film are laminate, and thus diaphragm portion 33 is formed on a top portion of semiconductor substrate 30. First electrode 36 made of titanium, platinum, and other materials, pyroelectric portion 37 made of a material having a higher dielectric constant, such as lead zirconate titanate, and second electrode 38 made of titanium, platinum, and other materials are sequentially formed on diaphragm portion 33 via insulating layer 31 such as a silicon oxide film.

### First Modification

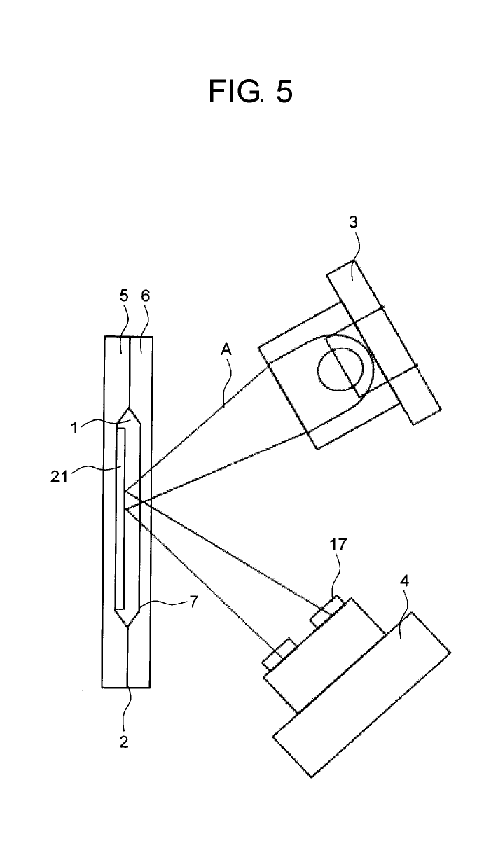

A disposition relationship of structure 2, light-emitting element 3, and photo-receptor element 4 may differ from the above described configuration. For example, as illustrated in FIG. 5, light-emitting element 3 and photo-receptor element 4 may be disposed so that light emitted from light-emitting element 3 is reflected by reflection film 21 in structure 2 to reach photo-receptor element 4. An example of a material for reflection film 21 is gold. With first substrate 5 made of a metallic material, a configuration without using reflection film 21 can be applied.

### Second Modification

The left view of FIG. 7 is a cross-sectional front view of structure 2 according to a second modification. The right view of FIG. 7 is a cross-sectional side view of structure 2 according to the second modification. In a structure configuring a sensor, as illustrated in FIG. 7, internal space 1 is formed by groove 7 of structure 2, and anti-reflection film 16 may be disposed on a side of groove 7, which lies closer to light-emitting element 3. Anti-reflection film 16 may further be disposed on another side lying closer to photo-receptor element 4. With anti-reflection film 16, through a surface reflection due to differences in refraction factor among a member configuring structure 2, air, and a fluid in internal space 1, an amount of light to reach photo-receptor element 4 can be prevented from reducing.

### Third Fourth Modifications

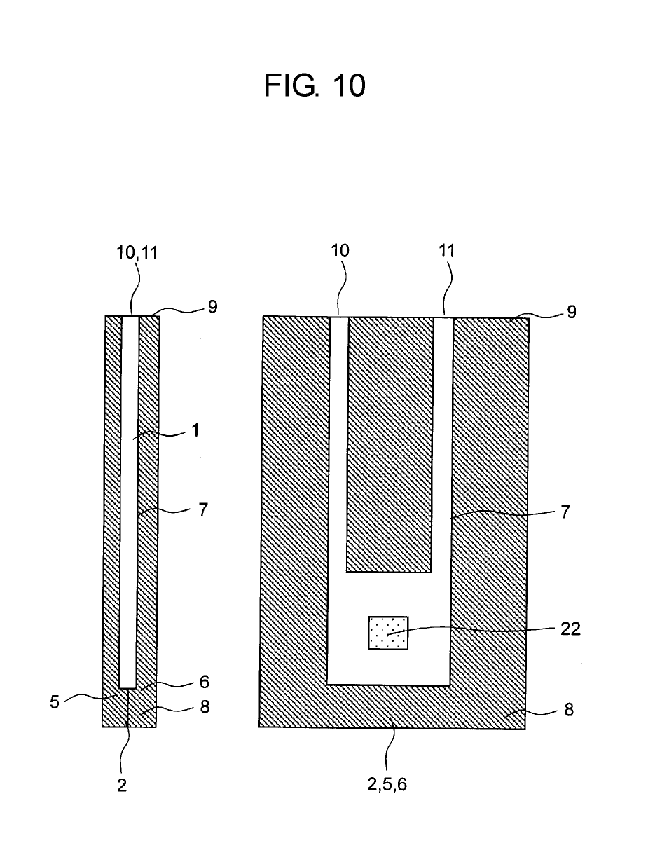

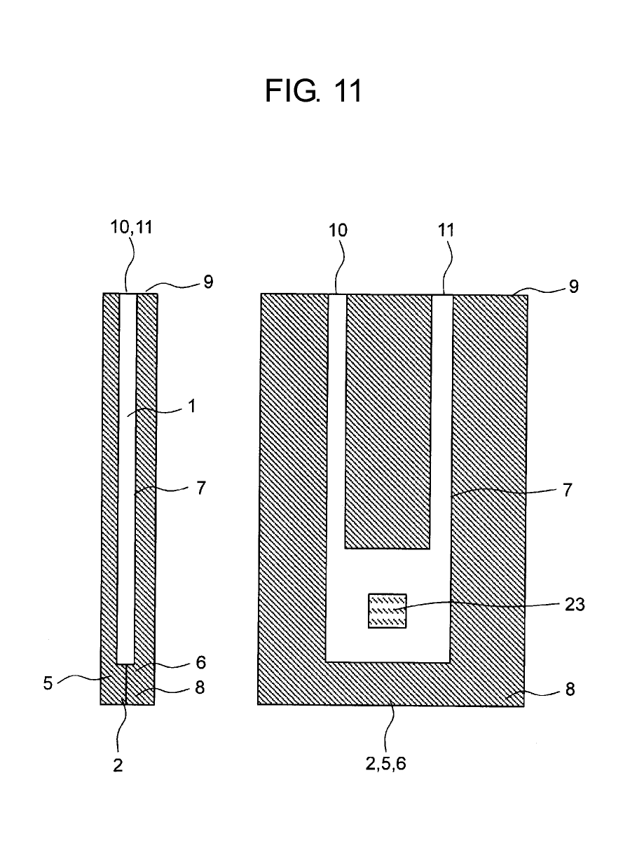

In addition, in a structure configuring a sensor, as illustrated in FIGS. 10, 11, structure 2 should preferably have a function capable of heating a detection target flowed into internal space 1. Specifically, in a third modification, as illustrated in FIG. 10, structure 2 includes member 22 for absorbing light emitted from light-emitting element 3. In addition, in a fourth modification, as illustrated in FIG. 11, structure 2 includes heater 23 for heating a detection target flowed into internal space 1. Since structure 2 has the function capable of heating a detection target, even if the sensor is small-sized and accordingly internal space 1 is narrowed, a detection target flowed into internal space 1 can be heated, and thus a convective flow is generated. This enables the detection target to easily enter and exit into and from internal space 1. As a result, the sensor with high sensitivity and high target substance selectivity can be provided. As a material for light absorbing member 22, diamond-like carbon (DLC) or a metallic oxide such as a ferrous oxide or a copper oxide may be used. Light absorbing member 22 for absorbing light should preferably be formed on an exterior of structure 2. However, light absorbing member 22 may be formed in internal space 1. In addition, a material for heater 23 should preferably be made of platinum, platinum rhodium, or another similar material. In addition, in terms of cost reduction, heater 23 should preferably be formed in a single layer.

### Fifth, Sixth, and Seventh Modifications

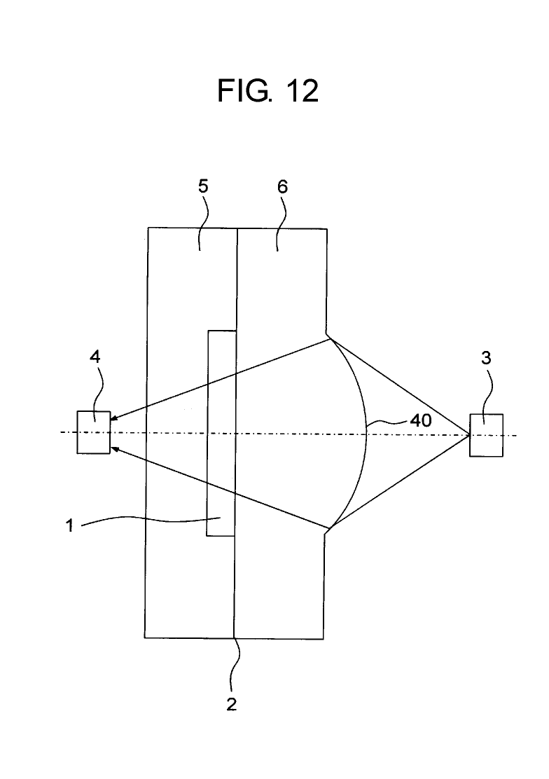

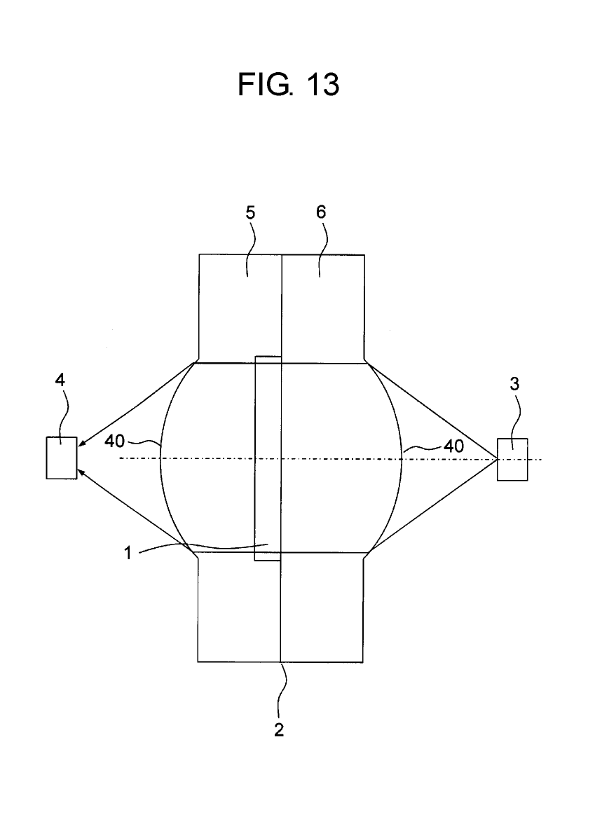

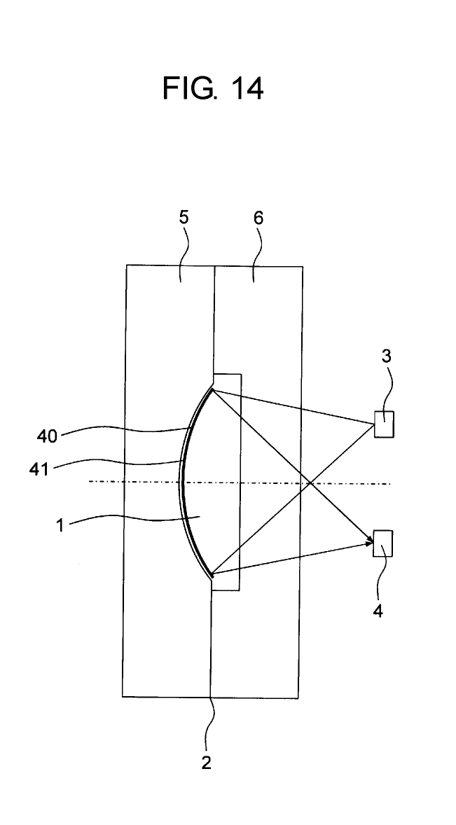

In addition, in a disposition relationship of a structure, a light-emitting element, and photo-receptor element configuring a sensor, in other modifications, as illustrated in FIGS. 12 to 14, lens portion 40 included in structure 2 allows light to be concentrated onto photo-receptor element 4. Specifically, in a fifth modification, as illustrated in FIG. 12, internal space 1 is made up of one or both of a first groove in first substrate 5 and a second groove in second substrate 6, and a face of second substrate 6, which lies opposite to internal space 1, has a convex portion. Note that, the convex portion can function as lens portion 40. Alternatively, in a sixth modification, as illustrated in FIG. 13, internal space 1 is made up of one or both of a first groove in first substrate 5 and a second groove in second substrate 6, and a face of first substrate 5, which lies opposite to internal space 1, and a face of second substrate 6, which lies opposite to internal space 1, each have a convex portion. Note that convex portions can function as lens 40. Alternatively, in a seventh modification, as illustrated in FIG. 14, internal space 1 is made up of a first groove in first substrate 5 and a second groove in second substrate 6, and the first groove is formed in an arc shape. Note that the first groove formed in the arc shape can function as lens portion 40.

In addition, as illustrated in FIG. 12, the convex portion that functions as a lens enables light that is emitted from light-emitting element 3 and reaches a peripheral portion of the convex portion to be concentrated onto photo-receptor element 4. Therefore, a light loss can be reduced, an amount of light reaching photo-receptor element 4 can be increased, and a sensor with high precision and high target substance selectivity can be provided. The convex portion may be formed by laminating a plurality of films, by grinding an area other than the convex portion, or by performing etching. A peripheral portion of the first groove should preferably be present inside the peripheral portion of the convex portion. This is because a larger amount of light emitted from light-emitting element 3 can securely pass through internal space 1.

In addition, as illustrated in FIG. 13, the convex portion that functions as a lens enables light that is emitted from light-emitting element 3 and reaches a peripheral portion of the convex portion to be concentrated onto photo-receptor element 4. Therefore, a light loss can be reduced, an amount of light reaching photo-receptor element 4 can be increased, and a sensor with high precision and high target substance selectivity can be provided. The convex portion may be formed by laminating a plurality of films, by grinding an area other than the convex portion, or by performing etching. A peripheral portion of the first groove should preferably be present inside the peripheral portion of the convex portion. This is because a larger amount of light emitted from light-emitting element 3 can securely pass through internal space 1.

In addition, as illustrated in FIG. 14, the first groove that functions as a concave portion enables light that is emitted from light-emitting element 3 and reaches a peripheral portion of the first groove to be concentrated onto photo-receptor element 4. Therefore, a light loss can be reduced, an amount of light reaching photo-receptor element 4 can be increased, and a sensor with high precision and high target substance selectivity can be provided. A peripheral portion of the second groove should preferably be present outside the peripheral portion of the first groove. This is because light concentrated by the first groove can securely pass through internal space 1. In addition, metal film 41 made up of gold, silver or other materials should preferably be disposed on a surface of the first groove. A metal having higher reflection rate enables an increase in a degree of concentration to photo-receptor element 4. A metal film may be formed on a surface, excluding a portion of the first groove, of first substrate 5 on which the first groove is formed. In addition, first substrate 5 may be formed of a metal having a higher reflection rate.

### INDUSTRIAL APPLICABILITY

With the sensor according to the present invention, a sensor with high sensitivity or high selectivity can be provided, and the sensor can be used as various sensors including fluid sensors. When a fluid is a fuel for a vehicle, density of a fuel component can be detected, and, for example, in an internal combustion engine, fuel economy can be improved, and exhaust emission can be reduced.

### REFERENCE MARKS IN THE DRAWINGS

1 internal space
2 structure
3 light-emitting element
4 photo-receptor element
5 first substrate
6 second substrate
7 groove
8 first end
9 second end
10 inflow port
11 outflow port
12 printed substrate
13 sealing body
14 reflecting mirror
16 anti-reflection film
17 optical filter
18 pipe bonding portion
19 wire
2020 pipe
21 reflection film
22 light absorbing member
23 heater
30 semiconductor substrate
31 insulating layer
32 recessed portion
33 diaphragm portion
34 light emission region
35 insulating layer
36 first electrode
37 pyroelectric portion
38 second electrode
40 lens portion
41 metal film
100 sensor

1. A sensor comprising:
a structure having an internal space into which a detection target is capable of flowing;
a light-emitting element; and
a photo-receptor element,
wherein the sensor is disposed so that light emitted from the light-emitting element passes through the internal space to reach the photo-receptor element, and
a wavelength of light emitted from the light-emitting element falls within a range from 2.5 μm to 15 μm inclusive.

2. The sensor according to claim 1, wherein a length of the internal space in a direction perpendicular to an extension direction of the structure is less than or equal to 1000 μm.

3. The sensor according to claim 1, wherein, in the internal space, an optical path length of the light is less than or equal to 1000 μm.

4. The sensor according to claim 1, wherein the structure includes a first substrate and a second substrate, the first substrate and the second substrate bonding at respective peripheral portions of the internal space.

5. (canceled)

6. (canceled)

7. The sensor according to claim 1, wherein the structure includes a first end in an extension direction of the internal space, and a second end on an opposite side of the structure from the first end, the first end being closed, the second end being open so that the detection target is capable of entering and exiting.

8. The sensor according to claim 7, wherein the second end includes an inflow port and an outflow port for the detection target.

9. The sensor according to claim 7, wherein a distance from the light-emitting element to the first end in the structure is shorter than a distance from the light-emitting element to the second end in the structure.

10. The sensor according to claim 1, further comprising a printed substrate,
wherein the light-emitting element, the photo-receptor element, and the structure are sealed with a sealing body, the sealing body being mounted on the printed substrate.

11. The sensor according to claim 1, wherein the light-emitting element includes two or more light sources each having a different wavelength.

12. The sensor according to claim 1, further comprising a reflecting mirror or a lens capable of concentrating light emitted from the light-emitting element.

13. The sensor according to claim 1, wherein the internal space is made up of a groove of the structure, the groove having an anti-reflection film on a side lying closer to the light-emitting element.

14. (canceled)

15. A sensor comprising:
a structure having an internal space into which a detection target is capable of flowing;
a light-emitting element; and
a photo-receptor element,
wherein the sensor is disposed so that light emitted from the light-emitting element passes through the internal space to reach the photo-receptor element, and
the structure is made up of a semiconductor substrate.

16. A sensor comprising:
a structure having an internal space into which a detection target is capable of flowing;
a light-emitting element; and
a photo-receptor element,
wherein the sensor is disposed so that light emitted from the light-emitting element passes through the internal space to reach the photo-receptor element, and
a length of the internal space in a direction perpendicular to an extension direction of the structure is less than or equal to 1000 μm.

17. The sensor according to claim 1, wherein the structure includes a member for absorbing light emitted from the light-emitting element.

18. The sensor according to claim 1, wherein the structure includes a heater for heating the detection target flowed into the internal space.

19. A sensor comprising:
a structure having an internal space into which a detection target is capable of flowing;
a light-emitting element; and
a photo-receptor element,
wherein the sensor is disposed so that light emitted from the light-emitting element passes through the internal space to reach the photo-receptor element, and
the structure has a function capable of heating the detection target flowed into the internal space.

20. The sensor according to claim 4, wherein
the internal space is made up of one or both of a first groove in the first substrate and a second groove in the second substrate, and
a face of the second substrate, which lies opposite to the internal space, has a convex portion.

21. The sensor according to claim 4, wherein the internal space is made up of one or both of a first groove in the first substrate and a second groove in the second substrate, and faces of the first substrate and the second substrate, which lie opposite to the internal space, each have a convex portion.

22. The sensor according to claim 4, wherein the internal space is made up of a first groove in the first substrate and a second groove in the second substrate, the first groove being formed in an arc shape.

23. A sensor comprising:
a structure having an internal space into which a detection target is capable of flowing;
a light-emitting element; and
a photo-receptor element,
wherein the sensor is disposed so that light emitted from the light-emitting element passes through the internal space to reach the photo-receptor element, and
the light is concentrated onto the photo-receptor element by a lens portion included in the structure.
